(19) United States
(12) Patent Application Publication
(10) Pub. No.: US 2024/0325378 A1
(43) Pub. Date: Oct. 3, 2024

(54) PERGOLIDE TREATMENT OF OCULAR CHEMICAL INJURY

(71) Applicant: University of Oregon, Eugene, OR (US)

(72) Inventors: Balamurali Ambati, Eugene, OR (US); Sangeetha Ravi Kumar, Springfield, OR (US); Kieley Trempy, Corvallis, OR (US); Xiaohui Zhang, San Marcos, CA (US)

(73) Assignee: University of Oregon, Eugene, OR (US)

(21) Appl. No.: 18/622,144

(22) Filed: Mar. 29, 2024

Related U.S. Application Data
(60) Provisional application No. 63/493,633, filed on Mar. 31, 2023.

(51) Int. Cl. A61K 31/48; A61K 9/00; A61K 9/08; A61P 27/02

(52) U.S. Cl. CPC A61K 31/48; A61K 9/0048; A61K 9/08; A61P 27/02

(57) ABSTRACT

Chemical injuries to the eye can cause permanent vision impairment or blindness, as well as chronic pain and dry eye syndrome. Disclosed herein is a method of treating an ocular chemical burn by administering an effective amount of pergolide to an eye of a subject, such as via a topical formulation. Further disclosed are compositions and kits used for that treatment, such as pergolide solutions, and pergolide eye drop formulations.

### CROSS-REFERENCE TO RELATED APPLICATION

This application claims priority to U.S. Provisional Application No. 63/493,633, filed Mar. 31, 2023, the entire contents of which are incorporated by reference in their entirety herein.

### FIELD

This relates to methods and compositions for treating ocular tissue with chemical burns using pergolide.

### BACKGROUND

Chemical injury of the eye is a sight threatening event (Galor 2022). These injuries cause extensive damage to the ocular surface, leading to corneal opacification and limbal stem cell deficiency (Dua 2020). Corneal anesthesia from these injuries can cause persistent epithelial defects and lead to neurotrophic keratitis (Xiang 2017). These injuries can also be associated with chronic ocular neuropathic pain and dry eye syndrome due to aberrant nerve regrowth during the healing process (Xiang 2017. Dua 2020, Gao 2017, Dieckmann 2017). Dry eye disease, which is frequently aggregated with ocular neuropathic pain, is associated with altered corneal nerve fibers (Patel 2021).

Standard treatment of corneal chemical injury consists of prophylactic antibiotics, vitamin C supplementation, and lubricating eye drops, which provide temporary pain relief (Dua 2020). Topical corticosteroids can control inflammation, however, they can hinder healing of the cornea (Dua 2020). In severe cases, limbal stem cell transplantation or whole cornea transplantation is required (Dua 2020). On the other hand, treatment for neuropathic ocular pain is variable and unsatisfactory, with options such as lubricating eye drops and neuro-regenerative eye drops (Galor 2015, Rauz et al. 2017). An example of neuro-regenerative eye drops is autologous or allogeneic serum. These provide endogenous factors such as NGF, insulin-like growth factor-1, epidermal growth factor, and substance P, which aid neural regeneration (Rauz et al. 2017). Serum eye drops are challenging to obtain because they require blood draws, and are not approved by the Food and Drug Administration (Rauz et al. 2017). Unfortunately, current treatments are limited in their ability to regenerate corneal tissue following a chemical injury.

### SUMMARY

Pergolide has previously been used systemically for its neuroregenerative effects in patients with Parkinson's disease. The present disclosure demonstrates the efficacy and safety of pergolide delivered as a topical eye drop in a mouse model of traumatic corneal alkali injury. Disclosed herein is a method of treating an ocular chemical burn, including administering an effective amount of pergolide to an eye of a subject having the ocular chemical burn, and compositions and kits used in that treatment, such as a pergolide solution.

The foregoing and other features of this disclosure will become more apparent from the following detailed description of several aspects which proceeds with reference to the accompanying figures.

### BRIEF DESCRIPTION OF THE FIGURES

The patent or application file contains at least one drawing executed in color. Copies of this patent or patent application publication with color drawing(s) will be provided by the Office upon request and payment of the necessary fec.

FIGS. 1A-1B show (1A) Schematic of the experimental methods. The high, medium, and low dose pergolide, vehicle, naïve, and NGF groups were followed for 3 weeks after corneal alkali injury. Wiping test was completed prior to the injury then once weekly for 3 weeks. At 3 weeks, eyes were examined and prepared as flat-mounts and hearts, livers, and kidneys were collected for paraffin embedding. Weight was measured on the day of the injury and at 3 weeks. (1B) Reconstructed 10× β-III tubulin stained whole-mount (1000 μm scale) of a pilot mouse 2 hours following corneal alkali injury shows the extent of the injury, demonstrating little involvement of the stromal nerves and marked difference in epithelial nerve density. The indicated areas are where measurements were taken for the endpoint mice. The central circle is slightly smaller than the actual area calculated to show the demarcation in the epithelial nerves between the injured and non-injured area.

FIGS. 2A-2B show the effect of pergolide on mouse pain behavior. Data represents the indicated week minus the baseline measurements; greater number of wipes is associated with more pain. (2A) Males showed increased pain in the high and medium dose pergolide and NGF groups largely at week 3 (N=5). (2B) Females showed a similar trend, though this response occurred earlier at week 2 (N=5). Data represents mean±std. dev. * P<0.05 vs. week 2, †P<0.05 vs. week 1 (two-way repeated measures ANOVA with multiple comparisons).

FIGS. 3A-3B show the effect of pergolide on corneal haze. (3A) Corneal haze grading showed decreased haze in the high dose pergolide and NGF groups 3 weeks post-injury (N=10; 5 male, 5 female). (3B) Representative images of each group at week 3 post-injury (males shown only due to no main effect of sex). Data represents mean±std. dev. * P<0.05 vs. naïve (two-way ANOVA with multiple comparisons).

FIGS. 4A-4C show the effect of pergolide on central corneal nerve fiber density 3 weeks following corneal alkali injury. (4A, 4B) Representative β-tubulin III stained flat-mounts of each group (10× scale: 1000 μm, 20× scale: 100 μm) (males shown only due to no main effect of sex). (4C) Central nerve density was higher in the high and medium dose pergolide as well as NGF groups (N=6; 3 male, 3 female). Data represents mean±std. dev. ** P<0.001 vs. low dose pergolide, vehicle, and naïve groups, *** P<0.001 vs, all groups (two-way ANOVA with multiple comparisons).

FIG. 5 shows the effect of pergolide on limbal nerve density 3 weeks following corneal alkali injury. Limbal nerve density was decreased in the low dose pergolide, vehicle, and naïve groups (N=6; 3 male, 3 female). Data represents mean±std. dev. ** P<0.001, *P<0.05 vs. low dose pergolide, vehicle, and naïve (two-way ANOVA with multiple comparisons).

### DETAILED DESCRIPTION

### I. Introduction

Corneal nerve injury is a significant clinical challenge after a chemical burn. Existing treatments are unsatisfactory. This disclosure demonstrates that topical pergolide can improve corneal nerve regeneration after chemical injury with alkali burn.

Alkali (0.75 M NaOH) injury was performed on the cornea of 1 eye per mouse (C57BL/6J, 5 males and 5 females per group). Mice were then given eye drops (10 μl) in the injured eye 3 times daily for 3 weeks; pergolide (600, 300, or 50 μg/ml as solution dissolved in a Marinosolv formulation), vehicle (Marinosolv only), or NGF (100 μg/ml). No treatment (injury only) and naïve (no injury/no treatment) groups did not receive drops. Pain behavior was tested weekly with the wiping test; corneal haze grading and body weight changes were assessed at 3 weeks. Heart and eye globes were collected at 3 weeks and corneal nerve fiber regeneration assessed with corneal flatmount antibody staining (anti-beta 3 tubulin).

Surprisingly, subjects presented with increased wiping behavior, consistent with patients who report pain during reinnervation of the cornea. Improvement in haze highlights the relationship between the corneal nerve, stromal, and epithelial cells, and their countercurrent exchange of factors. Behavioral response discrepancy between sexes without difference in other metrics may be related to pain processing. Topical pergolide improved corneal nerve regeneration.

### II. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a subject” includes singular or plural subject and can be considered equivalent to the phrase “at least one subject.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

About: Unless context indicated otherwise, “about” refers to plus or minus 5% of a reference value. For example, “about” 100 refers to 95 to 105.

Administration: To provide or give a subject an agent, such as a pergolide solution as described herein, by any effective route. Administration can be local or systemic. Exemplary routes of administration include, but are not limited to, injection (such as subcutaneous, intramuscular, intradermal, intraperitoneal, intrathecal, and intravenous), oral, sublingual, rectal, transdermal, intranasal, vaginal, inhalation, and topical (such as via an eye drop, or a spray) routes. In some examples, pergolide as provided herein (such as a pergolide solution) is administered locally to the eye via an eye drop (i.e., not administered systemically).

Autologous or Allogenic Eye Drops: Eye drops incorporating autologous or allogenic scrum such as peripheral blood serum, platelet rich plasma, or umbilical cord serum.

Chemical Burn: Damage to a subject's body caused by a corrosive or cytotoxic agent. Chemical burns can be caused by exposure to acids, alkalis (bases), and irritants. In some examples, chemical burns can be caused by biological toxins, such as anthrax toxin, or cytotoxic agents such as mustard gas. Chemical burns on mucosal surfaces, for example the ocular surface, can cause severe damage or blindness. The damage depends on the causative agent causing the chemical burn. Alkali agents cause liquefaction necrosis, which damages the protein matrix. Acidic agents cause coagulation necrosis. Alkali agents cause particularly severe damage, particularly in ocular tissues, in part due to begin lipophilic. Ocular chemical burns can result in limbal cell death, including limbal stem cells, corneal opacification or hazing, corneal neovascularization, symblepharon (abnormal adhesion between the bulbar and palpebral conjunctiva), abnormal intraocular pressure, trabecular meshwork damage, goblet cell loss, or lacrimal ductule scarring. The compositions and methods disclosed herein can be applied to any form of ocular chemical burn, including but not limited to, alkali chemical burns.

Common causative agents of chemical burns that can be treated with the disclosed compositions and methods include sulfuric acid, hydrofluoric acid, hydrochloric acid, acetic acid, formic acid, phosphoric acid, phenolic acid, chloroacetic acid, sodium hydroxide, calcium hydroxide, sodium hypochlorite, calcium hypochlorite, ammonia, phosphates, silicates, sodium carbonate, lithium hydride, bleaches, peroxides, chromates, and magnates.

Control: A reference standard. In some aspects, the control is a negative control. In other aspects, the control is a positive control. In some examples, a suitable control is a historical control or standard reference value or range of values (such as a previously tested control sample, such as a group of patients diagnosed with a disease or condition, for example ocular chemical burns that have a known prognosis or outcome, or a group of samples that represent baseline or normal values). In some examples, the control may be a subject not receiving treatment with an agent (e.g., the disclosed pergolide solution or pergolide eye drops) or receiving an alternative treatment, or a baseline reading of the subject prior to treatment with an agent.

A difference between a test sample and a control can be an increase or conversely a decrease. The difference can be a qualitative difference or a quantitative difference, for example a statistically significant difference. In some examples, a difference is an increase relative to a control, for example, an increase of at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%. In other examples, a difference is a decrease relative to a control, for example, a decrease of at least about 5%, at least about 10%, at least about 20%, at least about 30%, at least about 40%, at least about 50%, at least about 60%, at least about 70%, at least about 80%, at least about 90%, at least about 95%, at least about 98%, at least about 99%, or at least about 100%.

Cornea: The transparent front part of the eye that covers the iris, pupil, and anterior chamber. Together with the lens, the cornea refracts light, and as a result helps the eye to focus, accounting for approximately two-thirds of the eye's total optical power. The cornea has unmyelinated nerve endings sensitive to touch, temperature and chemicals; a touch of the cornea causes an involuntary reflex to close the eyelid. The cornea does not have blood vessels; it receives nutrients via diffusion from the tear fluid at the outside and the aqueous humor at the inside and also from neurotrophins supplied by nerve fibers that innervate it. In humans, the cornea generally has a diameter of about 11.5 mm and a thickness of 0.5-0.6 mm in the center and 0.6-0.8 mm at the periphery. The cornea has five layers; from the anterior to posterior these layers are the corneal epithelium. Bowman's layer, the corneal stroma, Descemet's membrane, and the corneal endothelium.

Corneal Haze: Clouding of corneal tissue, which can degrade vision. Corneal haze can be measured by methods such as those described in Roper-Hall M J. Thermal and chemical burns. Trans Ophthalmol Soc UK. 1965; 85:631-53.

Effective amount: The amount of an agent (such as pergolide, a pergolide solution, or other composition disclosed herein) that is sufficient to effect beneficial or desired results. An effective amount (also referred to as a therapeutically effective amount) may vary depending upon one or more of: the subject and disease condition being treated, the weight and age of the subject, the severity of the disease condition, the manner of administration and the like. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In one aspect, an “effective amount” of a therapeutic agent (e.g., a pergolide solution) is an amount sufficient to increase corneal nerve fiber density, increase limbal nerve density, improve vision, or combinations thereof, for example by at least 5%, at least 10%, at least 15%, at least 20%, at least 25%, at least 30%, at least 40%, at least 50%, at least 60%, at least 70%, at least 75%, at least 80%, at least 90%, at least 95%, or at least 99% (as compared to a suitable control, such as no administration of the therapeutic agent). In one aspect, an “effective amount” of a therapeutic agent (e.g., a pergolide solution disclosed herein, or a pergolide solution including a cycloplegic, a NSAID, a steroid, a hyaluronate or hydroxypropyl methylcellulose) is an amount sufficient to reduce signs or symptoms of ocular chemical burns such as corneal haze, or ocular pain for example by at least 5%, at least 10%, at least 15%, at least 20%, at least 25%, at least 30%, at least 40%, at least 50%, at least 60%, at least 70%, at least 75%, at least 80%, at least 90%, at least 95%, or at least 99% (as compared to a suitable control, such as no administration of the therapeutic agent). In some examples, combinations of these effects are achieved.

Fornix: Also called the Fornix conjunctiva, a highly vascularized mucous membrane tissue that forms a junction between the bulbar and palpebral conjunctive tissues. Palpebral conjunctive tissue lines the eyelid. Bulbar conjunctive tissue covers the anterior sclera of the eyeball.

Inflammation: A series of local tissue reactions that take place at a site of injury and have an immunological component. The injury may be due to trauma, lack of blood supply, hemorrhage. autoimmune attack, transplanted exogenous tissue or infection. This generalized response by the body includes the release of many components of the immune system (such as cytokines), attraction of cells to the site of the damage, swelling of tissue due to the release of fluid and other processes. Inflammation can be of an infectious or a non-infectious etiology. In the eye, inflammation produces vascular dilation, fluid leakage into extra-vascular spaces, migration of leukocytes and other cells.

Increase or Decrease: A positive or negative change, respectively, in quantity from a control value (such as a value representing no therapeutic agent). An increase is a positive change, such as an increase at least 25%, at least 50%, at least 100%, at least 200%, at least 300%, at least 400% or at least 500%, as compared to the control value. A decrease is a negative change, such as a decrease of at least 10%, at least 20%, at least 30%, at least 40%, at least 50%, at least 60%, at least 70%, at least 75%, at least 80%, at least 85%, at least 90%, at least 95%, at least 98%, at least 99%, or at least 100% decrease as compared to a control value. In some examples, the increase or decrease is statistically significant relative to a suitable control.

Pergolide: A dopamine receptor agonist, which has been sold under the brand names Permax® and Prascend®. In one example it has the chemical formula C₁₉H₂₆N₂S. PubChem CID 47811. The use of the term “pergolide” herein is inclusive of pergolide mesylate (PubChem CID: 47812). Pergolide has previously been used systemically for its neuroregenerative effects in patients with Parkinson's disease. Pergolide was removed from the United States market and is prescribed less frequently in other countries because of an association with cardiac valvulopathy. Pergolide and methods of preparation are discussed in U.S. Pat. No. 4,166,182.

Pharmaceutically acceptable carriers: Remington's Pharmaceutical Sciences. 23rd Edition, Academic Press, Elsevier, (2020), describes compositions and formulations suitable for pharmaceutical delivery of a therapeutic agent, such as pergolide disclosed herein.

In general, the nature of the carrier depends on the particular mode of administration being employed. For instance, parenteral formulations usually comprise injectable fluids that include pharmaceutically and physiologically acceptable fluids such as water, physiological saline, balanced salt solutions, aqueous dextrose, 5% human serum albumin, glycerol, or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. Supplementary active compounds can also be incorporated into the compositions. In some examples, Marinosolv® is a pharmaceutically acceptable carrier. In some examples, a liposomal microparticles is a pharmaceutically acceptable carrier.

Subject: A vertebrate, such as a mammal, for example a human. Mammals include, but are not limited to, murines, simians, humans, farm animals, sport animals, and pets. In one aspect, the subject is a non-human mammalian subject, such as a monkey or other non-human primate, mouse, rat, rabbit, pig, goat, sheep, dog, cat, horse, or cow. In some examples, the subject has ocular chemical burns, such as alkali ocular chemical burns, that can be treated using the pergolide or pergolide solution disclosed herein. In some examples, the subject is a laboratory animal/organism, such as a mouse, rabbit, or rat.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, blood and other clinical tests, and the like. In some examples, treatment with the disclosed methods results in a decrease in reported pain, a decrease in corneal hazing, or a decrease in signs and symptoms of ocular chemical burns. In some examples treatment with the disclosed methods results in an increase in nerve regeneration, such as an increase in central corneal nerve density, an increase in limbal nerve density, an increase in vision acuity, or a transient increase in pain as the corneal nerves regenerate.

### II. Overview

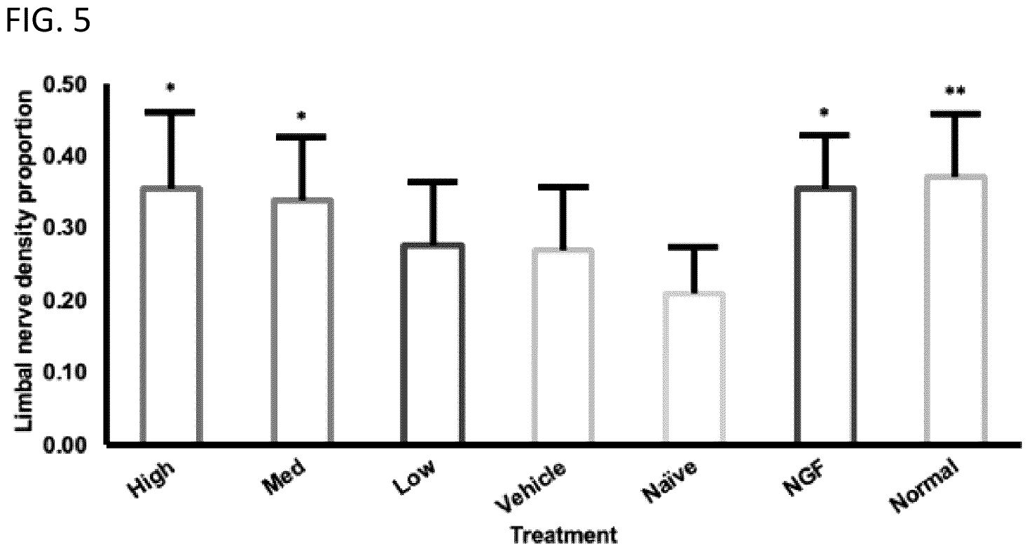

It is disclosed herein that pergolide improves corneal nerve density and symptoms in a mouse model of corneal alkali injury. Involvement of the limbal plexus was spared in pergolide treated animals compared to negative controls (FIG. 5). Though the initial alkali injury was only to the central cornea, it is possible that due to lack of sufficient treatment untreated groups experienced an inflammatory reaction involving the limbus. Limbal stem cell deficiency often occurs after chemical injury of the eye, even if the injury does not initially reach the limbal region (Dua 2020, Kadar 2011). There was comparable nerve density restoration between the medium (300 μg/ml) and high (600 μg/ml) doses of pergolide, comparable to (Zhang 2020). These doses were similar in the other metrics assessed herein demonstrating saturation of binding sites at the 300 μg/ml concentration. These results are consistent with the claim that pergolide exerts its effects by upregulating endogenous NGF (Ohta 2003, Zhang 2020).

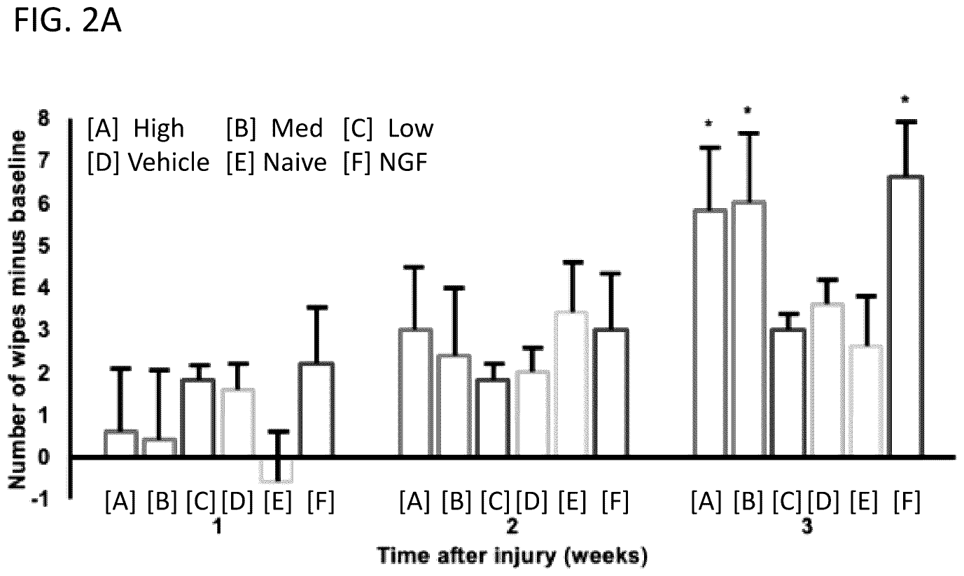

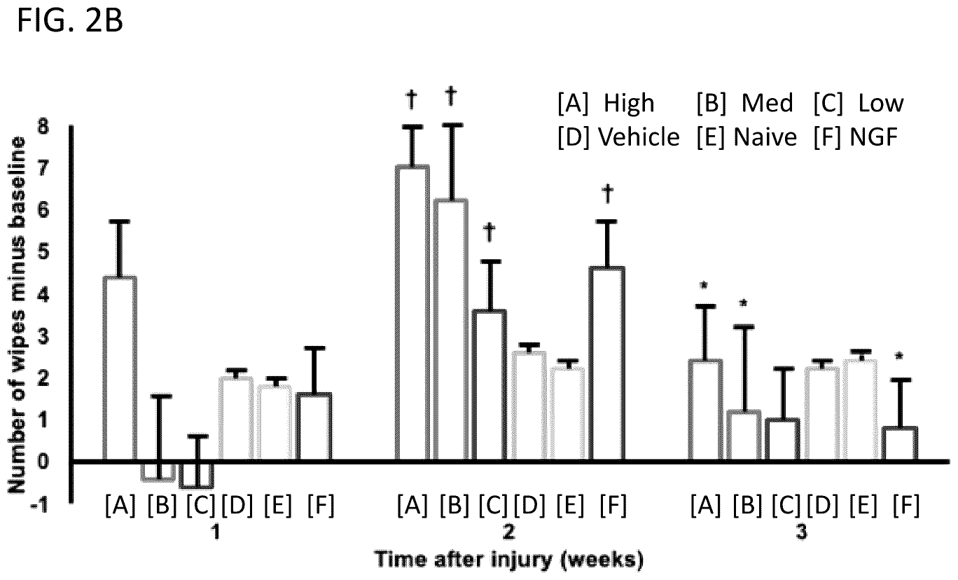

It was thought that pain behaviors would be decreased in pergolide treated subjects, but surprisingly, an increase in pain was observed in pergolide treated subjects (FIG. 2). Patients being treated with cenegermin experience increased pain while the nerves of the cornea reinnervate, followed by relief after completion of the treatment (di Zazzo 2020, Saricay 2022). Although there was a delayed pain response to therapy in males (FIG. 2A) compared to females (FIG. 2B), none of the other metrics (corneal haze, nerve fiber density) showed differences with regard to sex. This may indicate a difference in pain processing between males and females.

Improvement in corneal haze in the high dose pergolide treated subjects highlights the important relationship between the corneal nerve, stromal, and epithelial cells. Countercurrent exchange of factors occurs between these cells, resulting in nerve cell release of insulin-like growth factor-1 and epidermal growth factor. Uncoupling of this relationship causes a vicious positive feedback cycle; for example, injury to the corneal nerves reduces production of factors for the epithelial cells, resulting in decreased nerve growth factors, which further insults the nerves. Though there was decreased haze in the high dose pergolide treated subjects compared to the naïve group, there was no significance when compared to the low dose pergolide and vehicle groups. This may be due to these groups having minor therapeutic effect as a lubricating eye drop, which is a conventional therapy in corneal alkali injury (Dua 2020). These results indicate that the combination of (1) pergolide treatment, (2) in an eyedrop formulation has an additive effect in promoting corneal nerve regeneration, with the additional benefit of limiting pergolide bioavailability to ocular tissues.

Pergolide is a full agonist of dopamine (Newman-Tancredi D1 2002, Millan 2002) and 5-hydroxytryptamine (5-HT i.e., serotonin) (Newman-Tancredi 5-HT1 2002). Pergolide may elicit its effects through dopamine receptor agonism (Zhang 2020). It may be the case that pergolide also potentiates its effects through serotonin receptor agonism (Görnemann 2008), as serotonin has been implicated in the ocular pain pathways (Galor 2022). This is highlighted by the prevalence of dry eye disease (which is associated with altered corneal nerve fibers) in patients who take selective serotonin reuptake inhibitors (Patel, 2021, Galor 2022). Serotonin has been found in human tears (Martin 1994), corneal trigeminal ganglion neurons are responsive to serotonin (Veiga Moreira 2007), and its receptors are located in the cornea and conjunctiva (Grueb 2005, Potrebic 2003). Serotonin causes breakdown of phosphoinositide in the cornea through 5-HT2 receptors (Akhtar 1987) as well as promote adenyl cyclase and formation of PKA and cyclic AMP through 5-HT7 receptor agonism (Crider 2003, Grueb 2012). Serotonin increases chloride ion transport in corneal epithelium (Klyce 1982), but causes inhibition of its secretion in conjunctival epithelium (Alvarez. 2001). Serotonin also acts on the cornea by regulating the sensitization of nociceptors (Galor 2022). Serotonin binding leads to the release of substance P and calcitonin gene-related peptide, both which support corneal stromal and epithelial cells (Newman 5-HT1 2002, Galor 2022). Pergolide may activate serotonin binding in the cornea helping potentiate these effects. It is also possible that pergolide also exerts its effects through anti-inflammatory (Bendele 1991) and/or anti-oxidative stress activity (Uberti 2002).

The toxic cardiac effects of pergolide may be related to its agonism with serotonin receptors, which are localized in cardiomyocytes (Fortier 2019). In the examples disclosed herein, mice did not show significant weight loss or cardiac abnormalities, which has been observed in both human patients and animal studies involving use of systemic pergolide (Francis 1994, Droogmans 2007, Gilbert 2000, Fortier 2019, Simonds 2019). Treatment in the present study was topical and thus less bioavailable and occurring over a short course. Additionally, mouse retinas appeared unaffected. This was assessed because serotonin and dopamine can affect retinal vasculature and structure, respectively (Hayrch 1997, Seiple 2016).

Disclosed herein are methods for treating an ocular chemical burn, including administering an effective amount of pergolide to an eye of a subject having the ocular chemical burn. In some examples, the ocular chemical burn is caused by an alkali compound. In some examples, the subject is female. In some examples, the subject is male. In some examples, the subject is a mammal, such as a human. In some examples, the method includes selecting the subject having the ocular chemical burn. In some examples, the pergolide is administered as a pergolide solution via eye drop. In some examples, the effective amount of the pergolide solution administered is a drop of about 25 μl to about 60 μl, about 30 μl to about 55 μl, about 35 μl to about 50 μl, about 35 μl to about 45, or about 39 μl. In some examples, the effective amount of the pergolide solution administered is about 5 μl to about 7 μl on the cornea. In some examples, the effective amount of the pergolide solution administered is about 7 μl to about 10 μl on the eyelid fornix. In some examples, the concentration of pergolide in the pergolide solution is about 50 μg/ml to about 600 μg/ml to, about 50 μg/ml to about 300 μg/ml, about 50 μg/ml to about 200 μg/ml, about 50 μg/ml to about 100 μg/ml, about 600 μg/ml, about 300 μg/ml, about 120 μg/ml, or about 100 μg/ml.

In some examples, administering the effective amount of the pergolide solution does not result in cardiac valvulopathy in the subject. In some examples, administering the effective amount of the pergolide solution does not result in valvular fibrosis, significant cardiac glycosaminoglycan deposits, or cardiac myxoid changes in the subject. In some examples, administering the effective amount of the pergolide solution does not result in: weight loss in the subject, inflammatory cell infiltration in the subject's liver, necrosis in the subject's liver, lesions in the subject's liver, orthostatic hypotension in the subject, sustained hypotension in the subject, hallucinosis in the subject, pleuritis in the subject, pleural effusion in the subject, pleural fibrosis in the subject, pericarditis in the subject, pericardial effusion in the subject, or retroperitoneal fibrosis in the subject.

In some examples, the eye drop is administered 3 times a day, 2 times a day, or 1 time a day. In some examples, the eye drop is administered for 8 weeks or less, 7 weeks or less, 6 weeks or less, 5 weeks or less, 4 weeks or less, 3 weeks or less, 2 weeks or less, or 1 week or less. In some examples, the eye drop is administered for 1 week or more, 2 weeks or more, 3 weeks or more, 4 weeks or more, 5 weeks or more, 6 weeks or more, 7 weeks or more, or 8 weeks or more. In some examples, the eyedrop is administered for 1 to 2 weeks, 2 to 4 weeks, or 4 to 8 weeks. In some examples, the eye drop is administered beginning 1 month or more after the chemical burn occurred, 2 months or more after the chemical burn occurred, 3 months or more after the chemical burn occurred, 4 months or more after the chemical burn occurred, 5 months or more after the chemical burn occurred, or 6 months or more after the chemical burn occurred.

In some examples, administering the effective amount of pergolide reduces corneal haze, such as a reduction of at least 20%, at least 40%, at least 50%, at least 75%, at least 90%, or at least 95%. In some examples, administering the effective amount of pergolide increases central corneal nerve fiber density, such as an increase of at least 20%, at least 40%, at least 50%, at least 75%, at least 90%, at least 100%, at least 200%, at least 300% or at least 500%. In some examples, the central corneal nerve fiber density is increased within 1 week, within 2 weeks, or within 3 weeks following the administering, such as an increase of at least 20%, at least 40%, at least 50%, at least 75%, at least 90%, at least 100%, at least 200%, at least 300% or at least 500%. In some examples, administering the effective amount of pergolide prevents a decrease in limbal nerve density. In some examples, the limbal nerve density is increased within 1 week, within 2 weeks, or within 3 weeks following the administering, such as a reduction of at least 20%, at least 40%, at least 50%, at least 75%, at least 90%, or at least 95%.

In some examples, the pergolide or the pergolide solution is stable above 0° C., above 4.4° C., above 20° C., or above 40° C. In some examples, the pergolide or the pergolide solution does not degrade if stored for greater than 1 week, greater than 2 weeks, greater than 3 weeks, or greater than 4 weeks. In some examples the pergolide solution includes hyaluronate and/or hydroxypropyl methylcellulose.

In some examples, the subject was previously treated with one or more of vitamin C, a steroid, an antibiotic, lubricating eye drops, doxycycline, a tetracycline derivative, a non-aminoglycoside ocular antibiotic, erythromycin, a fluoroquinolone, mitomycin C, a medroxprogesterone eyedrop, a amniotic membrane eyedrop, a topical steroid, an autologous/allogenic eye drop, a citrate eye drop, an ascorbate eye drop, tenonplasty, amniotic membrane transplantation, punctal plug placement, a bandage contact lens, botulinum tarsorrhaphy, surgical tarsorrhaphy, limbal stem cell transplantation, deep anterior lamellar keratoplasty, penetrating keratoplasty, or keratoprosthesis. In some examples the subject did not previously receive ocular nerve grafting.

In some examples, the method further includes administering an effective amount of a cycloplegic, a NSAID, or a steroid to the eye of a subject having the ocular chemical burn. In some examples, the cycloplegic includes cyclopentolate or atropine. In some examples the NSAID includes bromfenac or ketorolac. In some examples the steroid includes prednisolone or dexamethasone.

Disclosed herein are kits that include a pergolide solution contained within a container configured to dispense a measured dosage of the pergolide solution (for example to the eye) and instructions for use. In some examples, the kit further includes one or more of a cycloplegic, a NSAID, and a steroid. In some examples, the cycloplegic is cyclopentolate or atropine. In some examples the NSAID is bromfenac or ketorolac. In some examples the steroid is prednisolone or dexamethasone.

### III. Compounds, Pharmaceutical Compositions Thereof, and Methods of Use

Disclosed herein are methods of treating a subject with one or more compounds that include pergolide (or a pharmaceutically acceptable salt thereof) provided herein. In some examples, the subject has ocular chemical burns. In some examples, the subject has ocular chemical burns caused by an alkali agent. In some examples the subject has corneal hazing, reduced central corneal nerve fiber density, or reduced limbal nerve density. In some examples the subject's eyes have been exposed to a chemical compound, and the subject may develop ocular chemical burns, or is suspected of having ocular chemical burns. In some examples, the pergolide used has the structure of formula 1:

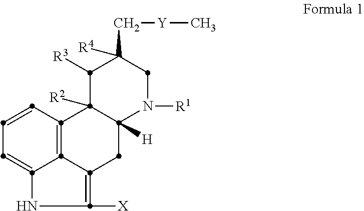

Wherein Y is O, SO, SO₂ or S, R¹ is ethyl, n-propyl, or allyl, X is H, Cl, or BR, and R², R³, and R⁴ when taken singly are hydrogen, and R² and R³, and R³ and R⁴, when taken together with the carbon atoms to which they are attached, form a double bond, and pharmaceutically-acceptable acid addition salts thereof. Exemplary compounds having this structure include: D-6-ethyl-8β-methylmercaptomethylergoline maleate, D-2-chloro-6-n-propyl-8β-methoxymethylergoline succinate, D-6-allyl-8β-methylmercaptomethylergoline hydrochloride, D-2-bromo-6-allyl-8β-methoxymethylergoline tartrate, D-6-n-propyl-8β-methylmercaptomethyl-9-ergolene hydrobromide, D-6-n-propyl-8-methoxymethyl-8-ergolene maleate, D-2-chloro-6-allyl-8β-methoxymethyl-9-ergolene benzoate, D-2-bromo-6-ethyl-8-methylmercaptomethyl-8-ergolene phosphate, D-6-ethyl-8β-methylsulfinylmethylergoline, D-6-n-propyl-8β-methylsulfonylmethyl-9-ergolene maleate, D-6-n-propyl-8-methylsulfinylmethyl-8-ergolene tartrate, D-2-chloro-6-allyl-8β-methylsulfimylergoline succinate, or D-2-bromo-6-allyl-8β-methylsulfinyl-9-ergolene.

This disclosure includes pharmaceutical compositions including at least one of the compounds described herein (e.g., pergolide or a pharmaceutically acceptable salt thereof) for use in human or veterinary medicine. Embodiments of pharmaceutical compositions include a pharmaceutically acceptable carrier and/or excipient and at least one of the disclosed compounds (e.g., pergolide or a pharmaceutically acceptable salt thereof).

The pharmaceutical compositions including one or more of the compounds disclosed herein (e.g., pergolide or a pharmaceutically acceptable salt thereof) may be formulated in a variety of ways depending, for example, on the mode of administration and/or the subject or disorder to be treated. For example, pharmaceutical compositions may be formulated as pharmaceutically acceptable salts. As an example, parenteral formulations may include a fluid for topical administration, such as an eyedrop. Pharmaceutically and physiologically acceptable fluid vehicles include water, physiological saline, other balanced salt solutions, aqueous dextrose, glycerol or the like. Excipients may include, for example, nonionic solubilizers, such as Cremophor®, or proteins, such as human serum albumin or plasma preparations. In some examples, the pharmaceutical composition to be administered may also contain non-toxic auxiliary substances, such as wetting or emulsifying agents, preservatives, and pH buffering agents and the like, for example, sodium acetate or sorbitan monolaurate.

Routes of administration include topical administration, such as via eyedrop. Topical preparations may include eye drops, ointments, sprays, and the like. To extend the time during which the compound is available to inhibit or treat a condition, the compound can be as a liposome or as a particulate system. The particulate system can be a microparticle, a microcapsule, a microsphere, a nanoparticle, a nanocapsule, or similar particle. The dosage form of the pharmaceutical composition can be determined, at least in part, by the mode of administration chosen.

In some embodiments, a carrier for preparing a formulation of a disclosed compound include Tween 80, glycerol, and a cyclodextrin (such as sulfobutylether-β-cyclodextrin (SBE-β-CD; Captisol®). In one example, the carrier includes 0.1% (v/v) Tween 80, and 99.9% (v/v) of 0.5% (w/v) of methylcellulose in water. In other embodiments, a carrier for preparing a formulation of a disclosed compound includes a non-ionic surfactant (e.g., caprylocaproyl polyoxyl-8 glycerides (Labrasol®), an oil (e.g., transesterified ethoxylated vegetable oil (e.g., Labrafil®), and a solubilizer (such as diethylene glycol monoethyl ether (e.g., Transcutol®). In a specific example, the carrier includes 40% (v/v) Labrasol, 40% (v/v) Labrafil, and 20% (v/v) Transcutol. Other carriers that can be used in formulations of the disclosed compounds (e.g., pergolide or a pharmaceutically acceptable salt thereof) include polyethylene glycol (e.g., PEG 400), propylene glycol; water (e.g., sterile water), and N,N-dimethylacetamide (DMA). Another exemplary carrier includes 5% (v/v) DMS), 2.5% (v/v) absolute ethanol, 2.5% (v/v) Solutol, and 90% saline. For ocular administration, the disclosed compounds (e.g., pergolide or a pharmaceutically acceptable salt thereof) may be formulated as a solution, emulsion, suspension, etc. suitable for administration to the eye. A variety of vehicles suitable for administering compounds to the eye are known in the art. Specific non-limiting examples are described in U.S. Pat. Nos. 6,261,547; 6,197,934; 6,056,950; 5,800,807; 5,776,445; 5,698,219; 5,521,222; 5,403,841; 5,077,033; 4,882,150; and 4,738,851. In some examples, the disclosed compounds (e.g., pergolide or a pharmaceutically acceptable salt thereof) are dissolved in Marinosolv® (manufactured by Marinomed®), as described in U.S. Pat. No. 11,510,859. In some examples, a disclosed compound, such as pergolide or a pharmaceutically acceptable salt thereof, is dissolved in an organic solvent, such as propylene glycol, followed by adding water containing a buffer (pH 5.2), saponin, and dexpanthenol, as described in Zhang et al., Invest. Ophth. & Vis. Sci. (January 2020) 61:4. In some examples, pergolide or a pharmaceutically acceptable salt thereof is prepared into drug-loaded liposomal microparticles by dissolving pergolide in chloroform, with 1,2-Distearoyl-sn-glycero-3-phosphocholine, cholesterol, and vitamin E, followed by perturbation to form a solution. The organic solvent can then be removed, and the product rehydrated with a carrier, such as PBS to produce pergolide-loaded liposomes, as described in Zhang et al., Invest. Ophth. & Vis. Sci. (January 2020) 61:4.

The disclosed compounds (e.g., that include pergolide or a pharmaceutically acceptable salt thereof) can be conveniently presented in unit dosage form. Such techniques include bringing into association the active ingredient (e.g., pergolide or a pharmaceutically acceptable salt thereof) and the pharmaceutical carrier(s) or excipient(s). The formulations can be included in unit-dose or multi-dose containers, for example, sealed ampoules and vials, or a dropper configured to dispense a measured volume, and may be stored in a dried condition requiring only the addition of a sterile liquid carrier, for example, water or saline immediately prior to use. In certain embodiments, unit dosage formulations are those containing a dose or unit, or an appropriate fraction thereof, of the administered ingredient.

The amount of the compound (e.g., pergolide or a pharmaceutically acceptable salt thereof) that is effective can depend on the nature of the disorder or condition to be treated (e.g., the extent of injury to the eye, or the agent that caused the chemical burn), as well as the stage of the disorder or condition. Effective amounts can be determined by in vitro studies, animal studies, and clinical techniques. The precise dose of the compounds to be included in the formulation can be decided according to the judgment of the health care practitioner and each subject's circumstances.

In one example, the dosage is administered as a number of drops per eye, with the drops having a particular concentration of pergolide solution. For example, one drop per eye, two drops per eye, three drops per eye, four drops per eye, five drops per eye, six drops per eye, seven drops per eye, eight drops per eye, nine drops per eye, or ten drops per eye. The dose can be administered at once (such as administering three drops at the same time) or administered over the course of the day (such as administering one drop in the morning, one drop at mid-day, and one drop in the evening). The eye drop can be administered one time a day, two times a day, three times a day, four times a day, five times a day, six times a day, seven times a day, eight times a day, nine times a day, or ten times a day. In some examples, the eye drop is administered no more than 3 times a day, such as 1, 2 or 3 times per day. The concentration of disclosed compound (e.g., pergolide or a pharmaceutically acceptable salt thereof) in the administration can vary. In some examples, the disclosed compound (e.g., pergolide or a pharmaceutically acceptable salt thereof) is present from about 50 μg/ml to about 600 μg/ml, about 50 μg/ml to about 300 μg/ml, about 50 μg/ml to about 200 μg/ml, about 50 μg/ml to about 100 μg/ml, about 100 μg/ml to about 600 μg/ml, about 100 μg/ml to about 300 μg/ml, about 100 μg/ml to about 200 μg/ml, about 150 μg/ml to about 600 μg/ml, about 150 μg/ml to about 300 μg/ml, about 150 μg/ml to about 200 μg/ml, about 200 μg/ml to about 600 μg/ml, about 200 μg/ml to about 300 μg/ml, about 200 μg/ml to about 250 μg/ml, or about 300 μg/ml to about 600 μg/ml. In some examples, the disclosed compound (e.g., pergolide or a pharmaceutically acceptable salt thereof) is present at greater than: 50 μg/ml, 100 μg/ml, 150 μg/ml, 200 μg/ml, 250 μg/ml, 300 μg/ml, 400 μg/ml, 500 μg/ml, 600 μg/ml, 700 μg/ml, 800 μg/ml, 900 μg/ml, or 1000 μg/ml. In some examples the disclosed compound (e.g., pergolide or a pharmaceutically acceptable salt thereof) is present at less than: 50 μg/ml, 100 μg/ml, 150 μg/ml, 200 μg/ml, 250 μg/ml, 300 μg/ml, 400 μg/ml, 500 μg/ml, 600 μg/ml, 700 μg/ml, 800 μg/ml, 900 μg/ml, or 1000 μg/ml. In some examples, the disclosed compound (e.g., pergolide or a pharmaceutically acceptable salt thereof) is present at about 50 μg/ml, about 100 μg/ml, about 120 μg/ml, about 150 μg/ml, about 200 μg/ml, about 250 μg/ml, about 300 μg/ml, about 400 μg/ml, about 500 μg/ml, or about 600 μg/ml. In some examples the disclosed compound (e.g., pergolide or a pharmaceutically acceptable salt thereof) is present at greater than: 60 μg/ml, 70 μg/ml, 80 μg/ml, 90 μg/ml, 100 μg/ml, 110 μg/ml, 120 μg/ml, 130 μg/ml, 140 μg/ml, 150 μg/ml, 160 μg/ml, 170 μg/ml, 180 μg/ml, 190 μg/ml, 200 μg/ml, 210 μg/ml, 220 μg/ml, 230 μg/ml, 240 μg/ml, 250 μg/ml, 260 μg/ml, 270 μg/ml, 280 μg/ml, or 290 μg/ml. In some examples the disclosed compound (e.g., pergolide or a pharmaceutically acceptable salt thereof) is present at less than: 60 μg/ml, 70 μg/ml, 80 μg/ml, 90 μg/ml, 100 μg/ml, 110 μg/ml, 120 μg/ml, 130 μg/ml, 140 μg/ml, 150 μg/ml, 160 μg/ml, 170 μg/ml, 180 μg/ml, 190 μg/ml, 200 μg/ml, 210 μg/ml, 220 μg/ml, 230 μg/ml, 240 μg/ml, 250 μg/ml, 260 μg/ml, 270 μg/ml, 280 μg/ml, or 290 μg/ml. In some examples, the disclosed compound (e.g., pergolide or a pharmaceutically acceptable salt thereof) is present from about 50 μg/ml to about 60 μg/ml, about 60 μg/ml to about 70 μg/ml, about 70 μg/ml to about 80 μg/ml, about 80 μg/ml to about 90 μg/ml, about 90 μg/ml to about 100 μg/ml, about 100 μg/ml to about 10 μg/ml, about 110 μg/ml to about 120 μg/ml, about 120 μg/ml to about 130 μg/ml, about 130 μg/ml to about 140 μg/ml, about 140 μg/ml to about 150 μg/ml, about 150 μg/ml to about 160 μg/ml, about 160 μg/ml to about 170 μg/ml, about 170 μg/ml to about 180 μg/ml, about 180 μg/ml to about 190 μg/ml, about 50 μg/ml to about 60 μg/ml, about 50 μg/ml to about 60 μg/ml, about 50 μg/ml to about 60 μg/ml, about 50 μg/ml to about 60 μg/ml

In some examples, the disclosed compound (e.g., pergolide or a pharmaceutically acceptable salt thereof) is dispensed in a measured drop, such as a drop of about 15 μl to about 70 μl, about 20 μl to about 65 μl, about 25 μl to about 60 μl, about 30 μl to about 55 μl, about 35 μl to about 50 μl, about 35 μl to about 45 μl, about 25 μl to about 40 μl, about 30 μl to about 35 μl, about 35 μl to about 55 μl, about 40 μl to about 50 μl, about 15 μl, about 20 μl, about 25 μl, about 30 μl, about 35 μl, about 36 μl, about 37 μl, about 38 μl, about 39 μl, about 40 μl, about 41 μl, about 42 μl, about 43 μl, about 44 μl, about 45 μl, about 50 μl, about 55 μl, or about 60 μl.

In some examples, the disclosed compound (e.g., pergolide or a pharmaceutically acceptable salt thereof) is dispensed such that a measured volume contacts an anatomical structure of the eye. In one example, about 2 μl to about 9 μl, about 5 μl to about 7 μl, or about 4 μl contacts the cornea. In one example, 5 μl to about 12 μl, about 7 μl to about 10 μl, 8 μl to about 9 μl, or about 8.5 μl contacts the eyelid fornix.

One or more doses of the compound (e.g., pergolide or a pharmaceutically acceptable salt thereof) can be administered to a subject. For example, the compound can be administered, every other day, twice per week, weekly, every other week, every three weeks, monthly, or less frequently. In some examples, the compound may be administered in cycles, for example, at a set interval (such as weekly or daily) for a set number of intervals, followed by a rest period, then repeated one or more times. In some examples the compound is administered three times a day, five days a week, with a rest period of two days per week. In some examples, the compound can be administered six times a day, five days a week, with a rest period of two days per week.

In some examples, the disclosed compound (e.g., pergolide or a pharmaceutically acceptable salt thereof) is administered to a subject soon after the chemical burn occurred, or is suspected to have occurred, such as within 10 minutes, within 30 minutes, within one hour, within 6 hours, within 12 hours, within 24 hours, within 2 days, within 3 days, within 5 days, or within 1 week. In some examples the disclosed compound is administered within 2 weeks, within 3 weeks, within 4 weeks, within 6 weeks, within 8 weeks, within 10 weeks, within 12 weeks, within 4 months, within 5 months, within 6 months, or within a year. In some examples the disclosed compound (e.g., pergolide or a pharmaceutically acceptable salt thereof) is administered beginning 12 hours, beginning 24 hours, beginning 1 week, beginning 2 weeks, beginning 3 weeks, beginning 4 weeks, beginning 2 months, beginning 3 months, beginning 4 months, beginning 5 months, beginning 6 months, beginning 7 months, beginning 8 months, beginning 9 months, beginning 10 months, beginning 11 months, or beginning 12 months or more after the chemical burn occurred or is suspected to have occurred.

The specific dose level and frequency of dosage for any particular subject may be varied and may depend upon a variety of factors, including the extent of the chemical burn being treated, the specific compound being administered, the agent that caused the chemical burn, the age, body weight, general health, and sex of the subject, mode and time of administration, and so on.

In some examples, the subject with ocular chemical burns is or was treated with another therapy, such as one or more of nerve growth factor, vitamin C, a steroid, an antibiotic, lubricating eye drops, doxycycline, a tetracycline derivative, a non-aminoglycoside ocular antibiotic, erythromycin, a fluoroquinolone, mitomycin C, a medroxprogesterone eyedrop, a amniotic membrane eyedrop, a topical steroid, an autologous/allogenic eye drop, a citrate eye drop, an ascorbate eye drop, tenonplasty, amniotic membrane transplantation, punctal plug placement, a bandage contact lens, botulinum tarsorrhaphy, surgical tarsorrhaphy, limbal stem cell transplantation, deep anterior lamellar keratoplasty, penetrating keratoplasty, or keratoprosthesis. In some examples, the subject receives or previously received ocular nerve grafting. In some examples, the subject was not previously treated with ocular nerve grafting.

In some examples the subject is treated in a combination therapy with cycloplegic (such as cyclopentolate or atropine), a NSAID (such as bromfenac or ketorolac), or a steroid (such as prednisolone or dexamethasone). The combination therapy can be formulated into the same administration route as the compounds disclosed herein (such as an eyedrop, or a spray) or administered separately (such as a separate injection, or oral formulation, or a separate eyedrop). A clinician can select an appropriate combination of additional treatments with the compounds provided herein, based on the ocular chemical burns being treated.

In some examples, treatment with the compounds disclosed herein reduces signs or symptoms of ocular chemical burns, relative to a suitable control, such as a subject with comparable burns, who is untreated, or is treated with an alternative therapy, such as a vehicle. Signs and symptoms of ocular chemical burns can include limbal cell death, corneal hazing, corneal neovascularization, symblepharon, increased intraocular pressure, decreased intraocular pressure, trabecular meshwork damage, goblet cell loss, lacrimal ductule scarring, dry eye syndrome, and/or pain. Administration of the compounds disclosed herein can reduce such signs or symptoms for example by at least 5%, at least 10%, at least 15%, at least 20%, at least 25%, at least 30%, at least 40%, at least 50%, at least 60%, at least 70%, at least 75%, at least 80%, at least 90%, at least 95%, or at least 99% (as compared to a suitable control, such as no administration of the therapeutic agent). In some examples, the reduction occurs at a specified time point, such as within 1 week, within 2 weeks, within 3 weeks, within 4 weeks, within 5 weeks, within 6 weeks, within 7 weeks, within 8 weeks, within 9 weeks, within 10 weeks, within 11 weeks, or within 12 weeks, of first administering the compounds disclosed herein. In some examples, signs or symptoms may worsen, for example pain can transiently worsen, but can be reduced following completion of a full treatment course as compared to a suitable control who was not administered the compounds disclosed herein. In some examples, administration of the compounds disclosed herein can increase corneal nerve fiber density, increase limbal nerve density, improve vision, or combinations thereof, for example by at least 5%, at least 10%, at least 15%, at least 20%, at least 25%, at least 30%, at least 40%, at least 50%, at least 60%, at least 70%, at least 75%, at least 80%, at least 90%, at least 95%, at least 100%, at least 110%, at least 120%, at least 130%, at least 140%, at least 150%, at least 160%, at least 170%, at least 180%, at least 190%, at least 200%, at least 300%, at least 400%, at least 500%, at least 600%, at least 700%, at least 800%, at least 900%, or at least at least 1000% (as compared to a suitable control, such as no administration of the therapeutic agent). In some examples, the increase occurs at a specified time point, such as within 1 week, within 2 weeks, within 3 weeks, within 4 weeks, within 5 weeks, within 6 weeks, within 7 weeks, within 8 weeks, within 9 weeks, within 10 weeks, within 11 weeks, or within 12 weeks, of first administering the compounds disclosed herein.

In some examples, the compositions disclosed herein are stable under a wide range of temperatures, such as above: −80° C., −20° C., 0° C., 1° C., 4° C., 20° C., 30° C., 40° C., 60° C., 80° C., 100° C., 150° C., or 200° C. In some examples, the compositions disclosed herein are stable under temperatures such as below: −80° C., −20° C., 0° C., 1° C., 4° C., 20° C., 30° C., 40° C., 60° C., 80° C., 100° C., 150° C., or 200° C. In some examples, the compositions disclosed herein are stable at room temperature such as 15° C.-25° C. In some examples, the compositions disclosed herein are stable at temperatures such as 0° C.-5° or −20° C.-0° C. In some examples, the compositions disclosed herein are stable when stored with exposure to light. In some examples, the compositions disclosed herein are stable when stored with exposure to sunlight. In some examples, the compositions disclosed herein are stable when stored in a dark environment. In some examples the compositions disclosed herein are stable under humidity conditions such as above: 0% RH, 10% RH, 20% RH, 30% RH, 40% RH, 50% RH, 60% RH, 70% RH, 80% RH, 90% RH, or 100% RH. In some examples the compositions disclosed herein are stable under humidity conditions such as below 1% RH, 10% RH, 20% RH, 30% RH, 40% RH, 50% RH, 60% RH, 70% RH, 80% RH, 90% RH, or 100% RH. In some examples, the compositions disclosed herein are stable, either in isolation, or as part of a solution, for a period of time, such as greater than 1 week, 2 weeks, 3 weeks, 4 weeks, 2 months, 3 months, 4 months, 5 months, 6 months, 1 year, 2 years, 3 years, 4 years, 5 years, 6 years, or longer. In some examples, the compositions have this extended stability despite being stored at room temperature (such as about 20° C.) and/or room humidity (such as 30% RH to 40% RH).

### IV. Kits

Also provided are compositions and kits that can be used with the disclosed methods. In some examples, the kit includes a pergolide (or a pharmaceutically acceptable salt thereof) solution contained within a container configured to dispense a measured dosage of the pergolide solution and instructions for use. In some examples, the kit includes a compound disclosed herein, within a container. In some examples, a container containing a pharmaceutically acceptable carrier or solvent, is also provided, configured to be mixed with the compound disclosed herein, such as pergolide. In some examples, the pharmaceutically acceptable carrier and the compound disclosed herein are included in a single container, but kept separate until a user mixes them, for example by breaking a divider. In some examples, the container is configured to dispense a measured spray. In some examples, the container is configured to dispense a measured drop. In some examples, instructions for use are provided with the kit.

In some examples, the kit further includes one or more of a cycloplegic, a NSAID, and a steroid. In some examples, the cycloplegic is cyclopentolate or atropine. In some examples the NSAID is bromfenac or ketorolac. In some examples the steroid is prednisolone or dexamethasone.

### V. Additional Aspects

Clause 1. A method of treating an ocular chemical burn, comprising administering an effective amount of pergolide to an eye of a subject having the ocular chemical burn.

Clause 2. The method of clause 1, further comprising selecting the subject having the ocular chemical burn.

Clause 3. The method of clause 1, wherein the pergolide is administered as a pergolide solution via eye drop.

Clause 4. The method of clause 3, wherein the effective amount of the pergolide solution administered is a drop of about 25 μl to about 60 μl, about 30 μl to about 55 μl, about 35 μl to about 50 μl, about 35 μl to about 45, or about 39 μl;
about 5 μl to about 7 μl on the cornea; or about 7 μl to about 10 μl on the eyelid fornix.

Clause 5. The method of clause 4, wherein the concentration of pergolide in the pergolide solution is about 50 μg/ml to about 600 μg/ml, about 50 μg/ml to about 300 μg/ml, about 50 μg/ml to about 200 μg/ml, about 50 μg/ml to about 100 μg/ml, about 600 μg/ml, about 300 μg/ml, about 120 μg/ml, or about 100 μg/ml.

Clause 6. The method of any one of clauses 1 to 5, wherein administering the effective amount of the pergolide solution does not result in cardiac valvulopathy in the subject.

Clause 7. The method of clause 6, wherein administering the effective amount of the pergolide solution does not result in valvular fibrosis, significant cardiac glycosaminoglycan deposits, or cardiac myxoid changes in the subject.

Clause 8. The method of any one of clauses 1 to 7, wherein administering the effective amount of the pergolide solution does not result in: weight loss in the subject, inflammatory cell infiltration in the subject's liver, necrosis in the subject's liver, lesions in the subject's liver, orthostatic hypotension in the subject, sustained hypotension in the subject, hallucinosis in the subject, pleuritis in the subject, pleural effusion in the subject, pleural fibrosis in the subject, pericarditis in the subject. pericardial effusion in the subject. or retroperitoneal fibrosis in the subject.

Clause 9. The method of any one of clauses 3 to 8, wherein the eye drop is administered 3 times a day, 2 times a day, or 1 time a day.

Clause 10. The method of any one of clauses 3 to 9, wherein the eye drop is administered for 1 or more weeks.

Clause 11. The method of any one of clauses 3 to 10, wherein the eye drop is administered beginning 1 month or more after the chemical burn occurred, 2 months or more after the chemical burn occurred, 3 months or more after the chemical burn occurred, 4 months or more after the chemical burn occurred, 5 months or more after the chemical burn occurred, or 6 months or more after the chemical burn occurred.

Clause 12. The method of any one of clauses 1 to 11, wherein the ocular chemical burn is caused by an alkali compound.

Clause 13. The method of any one of clauses 1 to 12, wherein administering the effective amount of pergolide reduces corneal haze.

Clause 14. The method of any one of clauses 1 to 13, wherein administering the effective amount of pergolide increases central corneal nerve fiber density.

Clause 15. The method of clause 14, wherein the central corneal nerve fiber density is increased within 1 week, within 2 weeks, or within 3 weeks following the administering.

Clause 16. The method of any one of clauses 1 to 15, wherein administering the effective amount of pergolide prevents a decrease in limbal nerve density.

Clause 17. The method of clause 16, wherein the limbal nerve density is increased within 1 week, within 2 weeks, or within 3 weeks following the administering.

Clause 18. The method of any one of clauses 1 to 17, wherein the pergolide or the pergolide solution is stable above 0° C., above 4.4° C., above 20° C., or above 40° C.

Clause 19. The method of any one of clauses 1 to 18, wherein the pergolide or the pergolide solution does not degrade if stored for greater than 1 week, greater than 2 weeks, greater than 3 weeks, or greater than 4 weeks.

Clause 20. The method of any one of clauses 1 to 19, wherein the subject was previously treated with one or more of vitamin C, a steroid, an antibiotic, lubricating eye drops, doxycycline, a tetracycline derivative, a non-aminoglycoside ocular antibiotic, erythromycin, a fluoroquinolone, mitomycin C, a medroxprogesterone eyedrop, a amniotic membrane eyedrop, a topical steroid, an autologous/allogenic eye drop, a citrate eye drop, an ascorbate eye drop, tenonplasty, amniotic membrane transplantation, punctal plug placement, a bandage contact lens, botulinum tarsorrhaphy, surgical tarsorrhaphy, limbal stem cell transplantation, deep anterior lamellar keratoplasty, penetrating keratoplasty, or keratoprosthesis.

Clause 21. The method of anyone of clauses 1 to 20, wherein the subject did not previously receive ocular nerve grafting.

Clause 22. The method of any one of clauses 1 to 21, further comprising administering an effective amount of a cycloplegic, a NSAID, a steroid, vitamin C, an antibiotic, lubricating eye drops, doxycycline, a tetracycline derivative, a non-aminoglycoside ocular antibiotic, erythromycin, a fluoroquinolone, mitomycin C, a medroxprogesterone eyedrop, a amniotic membrane eyedrop, an autologous/allogenic eye drop, a citrate eye drop, or an ascorbate eye drop to the eye of a subject having the ocular chemical burn.

Clause 23. The method of clause 22, wherein the cycloplegic comprises cyclopentolate or atropine, the NSAID comprises bromfenac or ketorolac, or the steroid comprises prednisolone or dexamethasone.

Clause 24. The method of any one of clauses 3 to 23, wherein the pergolide solution comprises hyaluronate or hydroxypropyl methylcellulose.

Clause 25. The method of any one of clauses 1 to 24, wherein the subject is female.

Clause 26. A kit, comprising a pergolide solution contained within a container configured to dispense a measured dosage of the pergolide solution and instructions for use.

### EXAMPLES

The following examples are provided to illustrate particular features of certain aspects of the disclosure, but the scope of the claims should not be limited to those features exemplified.

### Example 1

### Materials and Methods

This example illustrates the materials and methods used to carry out the following examples.

### Animal Care and Alkali Injury

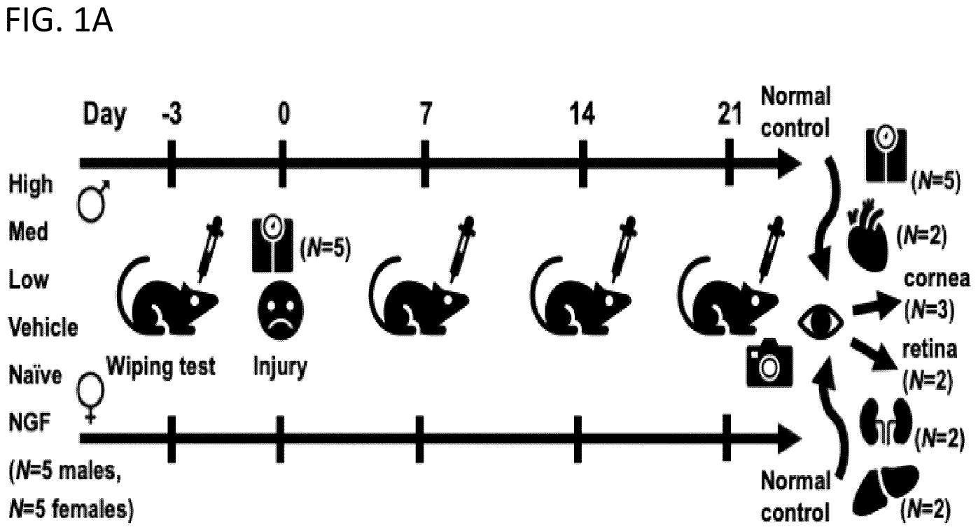

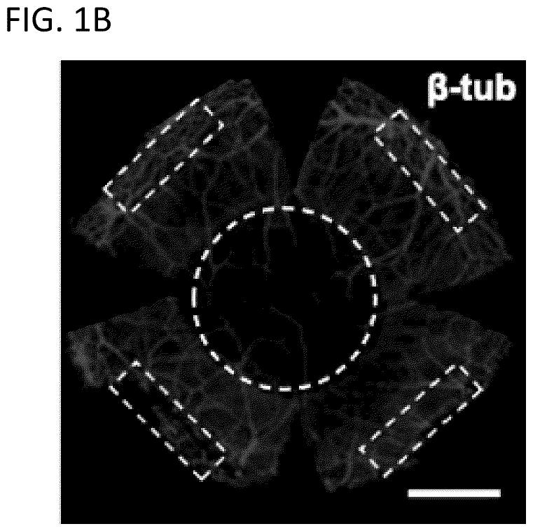

Seventy (35 male, 35 female) C57BL/6J mice (The Jackson Laboratory, Maine, US) were utilized (FIG. 1A). Mice were kept in a 12 hour light/12 hour dark cycle in groups of 5 with food and water ad libitum. Differences between males and females were assessed.

Mice were separated into 7 treatment groups with 10 mice per group (5 male, 5 female); high dose pergolide (600 μg/ml), medium dose pergolide (300 μg/ml), low dose pergolide (50 μg/ml), vehicle (solution only), NGF (100 μg/ml), naïve (injury and no treatment), and normal (no injury and no treatment). Preparation of pergolide and vehicle groups were done with Marinosolv® (Marinomed, Hovengasse, Korneuburg, AT) as described in (Siegl 2019 and Zhang 2020). Recombinant mouse NGF (R&D Systems, Minneapolis, MN, US) was prepared by dissolution in 1× phosphate-buffered saline (PBS). Both solutions were stored at 4 C according to the manufacturer's guidelines.

Mice were confirmed to have normal corneas prior to starting the study. Alkali corneal injury was done on one eye of each mouse for all groups excluding the normal group, in accordance with an established mouse model (Cho J 2019). Alkali-based injury was chosen as it is one of the most common chemical injuries to the eye (Dua 2020). In brief, sedation was achieved with ketamine:xylazine and confirmed with tail pinch. A 2 mm diameter filter paper disc was soaked in sodium hydroxide (0.75 M) then applied centrally on the cornea for 10 seconds. The disc was then removed and the ocular surface immediately irrigated with 10 ml of balanced sodium salts. The corneal surface was then lightly rubbed with the smooth side of forceps and irrigated in the same manner. None of the mice had eyelid or conjunctiva involvement of the injury. Erythromycin ointment (Perrigo, Dublin, IE) was then applied to the exterior of both eyes. Mice were placed on a water heating pad, monitored until fully awake, then returned to their cage.

All groups except the no treatment groups received their respective eye drops (10 μl per eye drop) three times daily (5 out of 7 days each week) in the injured eye for 3 weeks following injury. Mice were held for 10 seconds with the eye drop on their eye before being returned to their cage. Mice were sacrificed by cervical dislocation after week 3.

### Pain Behavior

Wiping test was chosen to assess ocular pain behavior and was done once weekly (at the same time), in accordance with previously established protocols (N=10; 5 male, 5 female) (Tashiro et al. 2010, Cho J et al. 2019). Testing was performed in a clear open-top cylinder surrounded by 2 mirrors and a video recorder in front of the cylinder. Mice were habituated to the cylinder prior to the start of the study. The test consisted of applying 10 μl of sodium chloride (2 M) to the surface of the study cornea for 10 seconds, blotting excess away, then recording the mouse in the cylinder for 30 seconds. This molarity was chosen as it has been shown to not induce ocular inflammation while still eliciting consistent behaviors (Tashiro et al. 2010). A different pipette tip was used for each mouse. Sudden movements and loud noises were avoided during testing so as to not distract the subjects. An eye wipe was defined as a purposeful wipe by the ipsilateral forelimb across the eye or resulting in eye closure. Cage changes were not done within 3 days of each test, and when cage changes occurred it happened for each group simultaneously. Eye wiping was assessed at baseline (3 days prior to injury) and on post-injury weeks 1, 2, and 3.

### Corneal Haze Grading

Corneal haze grading (N=10; 5 male, 5 female) was done 3 weeks following the initial injury under a microscope (Leica, Wetzlar, Hesse, DE) according to the Roper-Hall classification, with “0” as clear, “1” as epithelial damage, “2” as hazy but iris details seen, “3” as complete epithelial loss and stromal haze obscuring iris, and “4” as opaque (Roper-Hall 1965).

### Immunofluorescence Staining

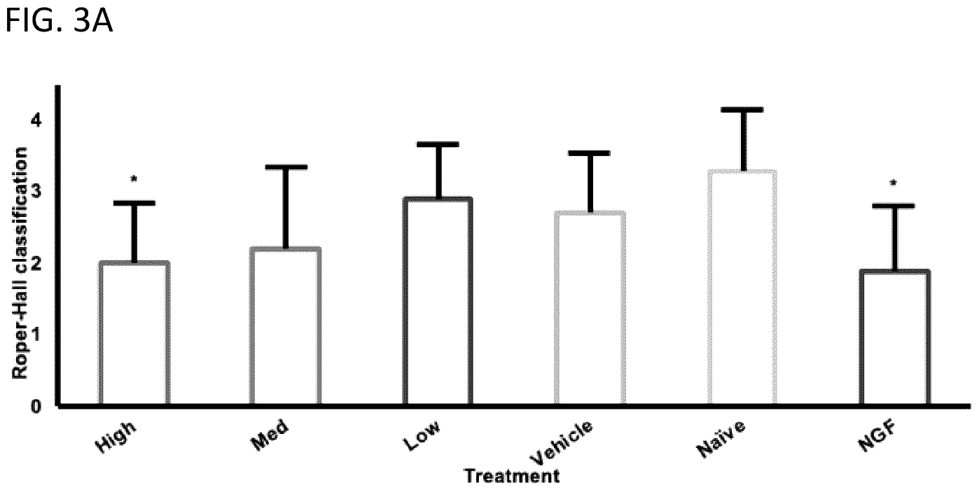

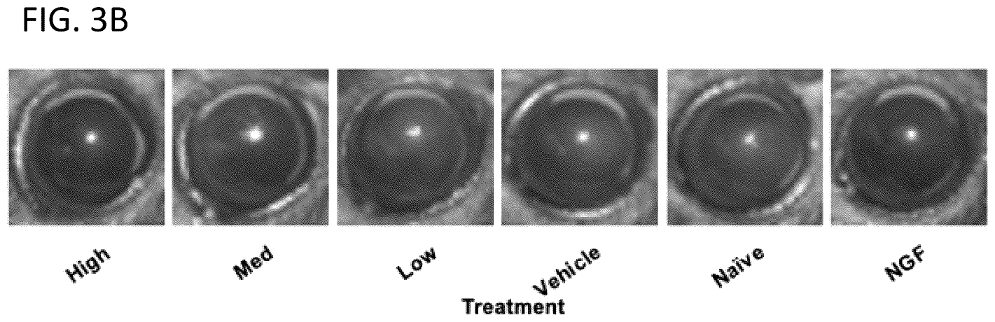

Flat-mounts of mouse corneas were prepared and corneal nerve density was analyzed as previously described (N=6; 3 male, 3 female) (Zhang 2020) (FIG. 3A). In brief, following euthanasia the study eye was enucleated from each mouse and the other portions of the eye removed to expose the cornea. Corneas were fixed in 100% acetone shaking for 1 hour, washed with 1×PBS, blocked in 1×PBS-3% bovine serum albumin for 1 hour, then incubated overnight at 4° C. with nonconjugated p III tubulin polyclonal rabbit antibody (Abcam, Cambridge, ENG, UK) at a concentration of 1:200. Corneas were then incubated for 1 hour with secondary antibody Alexa Fluor™ 546 (Thermo Fisher, Waltham, MA, US), washed with 1×PBS, and mounted with anti-fading agent gel (Electron Microscopy Sciences, Hatfield, PA, US).

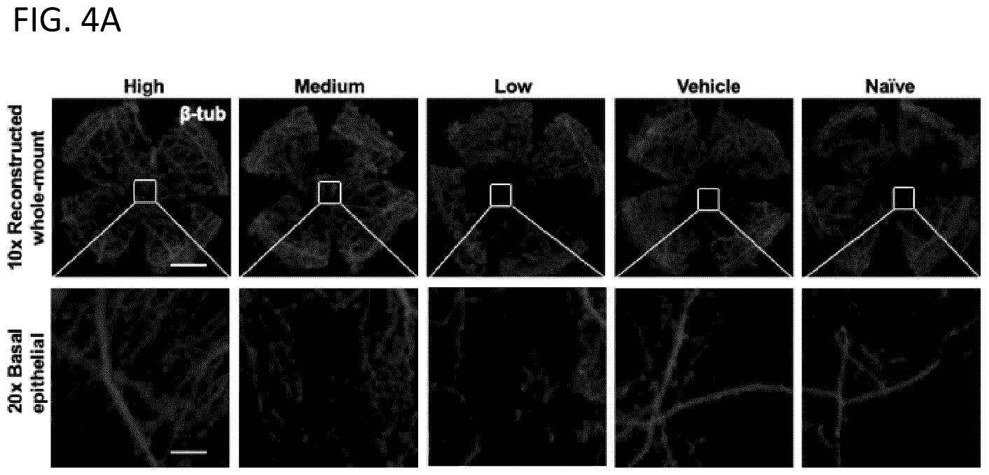

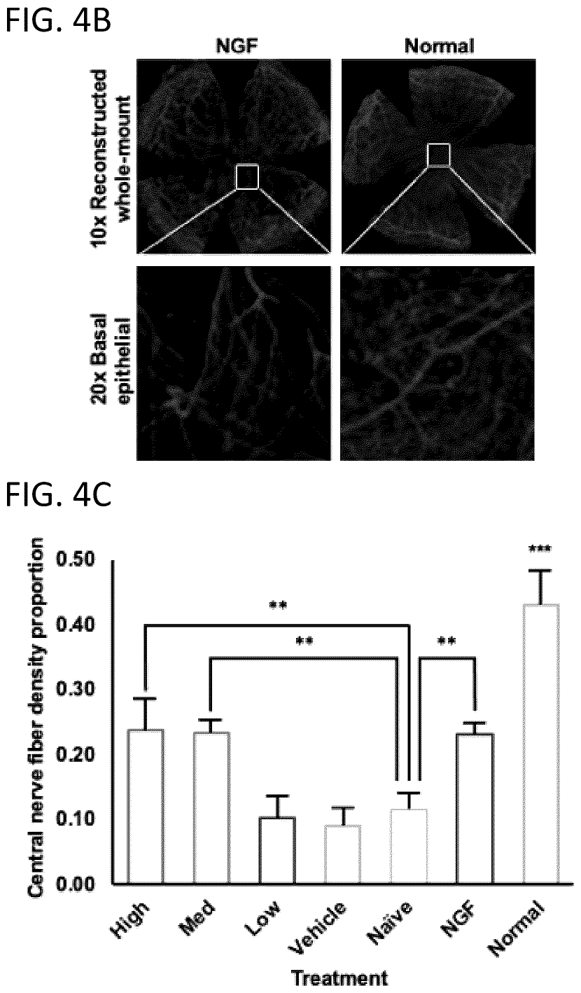

EVOS fluorescence microscope (Life Technologies, Carlsbad, CA, US) was used to create reconstructed whole-mount images and take images at the basal epithelial level in the central area of the injury. ImageJ was used to calculate central corneal nerve density as the proportion of area with β-tubulin III staining in the 2000×2000 μm circular area of the initial injury (FIG. 4C). Four measurements (one from each quadrant of the flat-mount) were collected to get an average of the limbal nerve density (FIG. 4C) (Daeschler 2022).

### Histopathology and Toxicity

Change in mouse weight was compared from baseline to 3 weeks post-injury, and weight in the pergolide groups was compared to the non-pergolide groups (N=10; 5 male, 5 female). Following euthanasia, the heart and eyes were removed from the high dose pergolide and vehicle groups and histopathology was assessed using previously described protocols.

In brief, after excision, hearts, livers, and kidneys were fixed in formalin for 24 hours then embedded in paraffin and cut in the coronal and transverse planes (N=4; 2 male, 2 female) (Droogmans 2007 & 2009). Eyes were fixed in Davidson's solution for 24 hours then placed in 70% ethanol and processed prior then embedded in paraffin and cut in the sagittal plane (N=4; 2 male, 2 female) (Chidlow 2011). Hematoxylin and eosin were used to stain the slides (Cardiff 2014), then the EVOS fluorescence microscope was used to capture images.

### Statistical Analysis

Sample sizes were chosen based on similar studies (Cho 2019, Zhang 2020). Analyses were performed using SPSS version 29 (IBM, Chicago, IL, US). Data are presented as means and standard deviations. Overall differences were analyzed with two-way analysis of variance (ANOVA) (repeated measures for data taken at multiple timepoints), and significance between groups was established with Tukey's multiple comparisons test. Alpha level was set at P value of less than 0.05. Normality was confirmed with Shapiro-Wilk tests.

### Example 2

### Pain Behaviors

This example illustrates wiping behavior in mice following pergolide treatment.

There was a main effect of sex and week(s) post-injury (P<0.001) with regard to wiping. Male wiping behavior is depicted in FIG. 2A. Female wiping behavior is depicted in FIG. 2B. In the males, wiping was increased in the high (5.8±4.9 wipes) and medium (6.0±2.3 wipes) dose pergolide and NGF (6.6±5.6 wipes) groups at week 3 compared to week 1 (P=0.007, P=0.004, P=0.021 respectively). In the females, wiping was also heightened in the high (7.0±5.8 wipes) and medium (6.2±5.5 wipes) dose pergolide and NGF (4.6±5.1 wipes) groups though this occurred at week 2 compared to week 1 (P=0.016, P<0.001, P=0.019, respectively). In the females, wiping then decreased in these groups at week 3 compared to 2 (P=0.025, P=0.009, P=0.045, respectively).

This unexpected increase in pain can be explained by increased healing may be related to improved corneal nerve regeneration. An improvement in corneal nerve sensation may induce some amount of pain in the healing process after chemical burn. A total lack of nerve sensation predisposes subjects to infections, corneal melting, and further injury.

### Example 3

### Corneal Haze Grading

This example illustrates corneal haze grading following pergolide treatment.

At 3 weeks post injury, there was a main effect of treatment on corneal haze (P=0.004). There was no main effect observed for sex (P=0.483). Haze was less in the high dose pergolide (2.0±0.8, P=0.029) and NGF (1.9±0.9, P=0.007) groups compared to the naïve group (3.3±0.8) (FIGS. 3A, 3B). Medium dose pergolide group (2.2±1.1, P=0.095) trended towards less haze compared to the naïve group.

### Example 4

### Central Corneal Nerve Density

This example illustrates central corneal nerve density following pergolide treatment.

FIGS. 4A and 4B shows β-tubulin III staining across treatment groups. FIG. 4C depicts mean central nerve density across treatment groups. There was a main effect of treatment (P<0.001) but not of sex (P=0.972). Central corneal nerve density was markedly less in all groups compared to the normal control (0.43±0.05, P<0.001) (FIG. 4). Medium (0.23±0.02) and high (0.24±0.05) dose pergolide as well as NGF (0.23±0.02) showed higher central nerve density compared to the low dose pergolide (0.10±0.03), vehicle (0.09±0.03), and naïve (0.11±0.03) groups (P<0.001).

### Example 5

### Limbal Nerve Density

This example illustrates how pergolide treatment prevented a decrease in limbal nerve density following ocular chemical injury.

FIG. 5 depicts limbal nerve density 3 weeks following injury, across treatment groups. There was a main effect of treatment (P<0.001) but not of sex (P=0.197) with respect to limbal nerve density. Normal control (0.37±0.09) had higher limbal nerve density than the low dose pergolide (0.28±0.09, P<0.001), vehicle (0.27±0.09, P<0.001), and naïve groups (0.21±0.06, P<0.001), but was no different when compared to high (0.35±0.11, P=0.506) and medium (0.34±0.08, P=0.206) dose pergolide and NGF (0.36±0.08, P=0.525).

It was not previously appreciated that chemical insult to the central cornea could affect limbal nerve density. The limbus was not directly injured in this model, yet untreated (‘Naïve’) and vehicle treated animals presented with limbal nerve injury following sodium hydroxide exposure to the central cornea. It is possible that these untreated groups experienced an inflammatory reaction following central corneal injury, which subsequently involved the limbus. Pergolide treatment effectively prevented this previously unappreciated limbal nerve injury. Treatment with 600 μg/ml and 300 μg/ml pergolide resulted in limbal nerve density comparable to uninjured mice (‘Normal’).

### Example 6

### Side Effects of Topical Pergolide

Following treatment with high, medium, and low dose pergolide, hearts did not show presence of valvular fibrosis, glycosaminoglycans deposits/myxoid changes, chondroid metaplasia, or other abnormal pathology compared to the vehicle group. Retinas, livers, and kidneys also did not show abnormal morphology.

No significant weight loss was observed in any of the groups between baseline and 3 weeks post-injury. Two mice in the study exhibited minor weight loss (in the medium dose and vehicle groups) and this amount was negligible at about 1% and 0.5% of their initial body weight, respectively.

### Example 7

### Pergolide Dosage and Long Term Effects

Pergolide will be administered in eyedrops at concentrations ranging from 50 μg/ml to 600 μg/ml. Pergolide will be tested on mice with alkali corneal injury as described in Example 1. Pergolide drops will be administered 2 times a day and/or 3 times a day. In one example, pergolide will be administered at a concentration of 120 μg/ml. NGF expression will be evaluated in eye tissues following pergolide treatment.

Animals will also be treated with topical pergolide over a longer period, and in a greater number of animal subjects. Animals will be weighed, and have organs such as heart, liver, and kidneys removed and assessed via histopathology to assess for systemic effects.

### Example 8

### Corneal Esthesiometry

Pergolide will be administered to mice with alkali corneal injury as described in Example 1, and subjects will be assessed with corneal esthesiometry at 1, 2, and 3 weeks concurrent with a wiping test as described in Example 1 (just prior to the wiping test as the saline solution may impact sensation).

It may be that baseline pain measurements are not painful to the mice because they did not have a corneal injury, while week 1 post-injury mice had a lack of response due to sensation loss. Esthesiometry will be assessed to determine if mice show decreased or absent sensation. Corneal anesthesia has been demonstrated in a similar study of corneal alkali injury in mice for up to 3 weeks (Xiang 2017). Therefore, esthesiometry would likely show normal sensation.

### Example 9

### Improved Stability

The stability of pergolide, pergolide solutions, and pharmaceutical compositions thereof will be assessed in comparison to alternative treatments, such as autologous/allogenic eye drop or NGF, under a range of storage conditions.

Pergolide will be incorporated into a solution as described in example 1, and placed in storage for between one week and one year. The pergolide solution will be stored at temperature ranges such as −20° C.-0° C., as 0° C.-5°⁰ and −20° C.-0° C. The pergolide solution will be stored such that it is not exposed to light, such that it is exposed to indoor lighting, and such that it is exposed to sunlight. Following storage, stability of pergolide in solution will be assessed by liquid chromatography-mass spectrometry (LC-MS), using the methods of Kumar et al., Mol. And Cel. Biochem. (2008) 333:299-310, incorporated by reference herein in its entirety.

### REFERENCES

1. Zhang X, Muddana S, Kumar S R, Burton J N, Labroo P, Shea J, et al. Topical pergolide enhance corneal nerve regrowth following induced corneal abrasion. Investig Ophthalmol Vis Sci. 2020; 61(1):4.
2. Fortier J H, Pizzarotti B, Shaw R E, Levy R J, Ferrari G, Grau J. Drug-associated valvular heart diseases and serotonin-related pathways: A meta-analysis. Heart. 2019; 105(15):1140-8.
3. Droogmans S, Franken P R, Garbar C, Weytjens C, Cosyns B, Lahoutte T, et al. In vivo model of drug-induced valvular heart disease in rats: Pergolide-induced valvular heart disease demonstrated with echocardiography and correlation with pathology. Eur Heart J. 2007; 28(17):2156-62.
4. Grnemann T, Hübner H, Gmeiner P, Horowski R, Latte K P, Flieger M, Pertz H H. Characterization of the molecular fragment that is responsible for agonism of pergolide at serotonin 5-hydroxytryptamine_{2B} and 5-hydroxytryptamine_{2A} receptors. J Pharmacol Exp Ther. 2008 Mar. 1; 324(3):1136-45.
5. Fleeman N, Mahon J, Nevitt S, Duarte R, Boland A, Kotas E, Dundar Y, McEntee J, Ahmad S. Cenegermin for treating neurotrophic keratitis: an evidence review group perspective of a NICE single technology appraisal. PharmacoEconomics. 2019; 3(4):453-61.
6. Cho J, Bell N, Botzet G, Vora P, Fowler B J, Donahue R, et al. Latent sensitization in a mouse model of ocular neuropathic pain. Transl Vis Sci Technol. 2019; 8(2):6.
7. Galor A, Levitt R C, Felix E R, Martin E R, Sarantopoulos C D. Neuropathic ocular pain: an important yet underevaluated feature of dry eye. Eye. 2015; 29:301-312.
8. Rauz S, Koay S Y, Foot B, Kaye S B, Figueiredo F, Burdon M A, Dancey E, Chandrasekar A, Lomas R. The Royal College of Ophthalmologists guidelines on serum eye drops for the treatment of severe ocular surface disease: full report. Eye. 2017:1-6.
10. Ji Y W, Kang H G, Song J S, Jun J W, Han K, Kim T-im, et al. The dopaminergic neuronal system regulates the inflammatory status of mouse lacrimal glands in dry eye disease. Investig Ophthalmol Vis Sci. 2021; 62(4):14.
11. Saricay L Y, Bayraktutar B N, Lilley J, Mah F S, Massaro-Giordano M, et al. Efficacy of recombinant human nerve growth factor in stage 1 neurotrophic keratopathy. Ophthalmol. 2022; 129(9).
12. di Zazzo A, Varacalli G, Mori T, Coassin M. Long-term restoration of corneal sensitivity in neurotrophic keratopathy after rhNGF treatment. Eur J Ophthalmol. 2020; 32(1).
13. Dieckmann G, Goyal S, Hamrah P. Neuropathic corneal pain: approaches for management. Ophthalmol. 2017; 124(11):S34-47.
14. Chidlow G, Daymon M, Wood J P, Casson R J. Localization of a wide-ranging panel of antigens in the rat retina by immunohistochemistry: comparison of Davidson's solution and formalin as fixatives. J Histochem Cytochem. 2011; 59(10):884-98.
15. Galor A, Hamrah P, Haque S, Attal N, Labetoulle M. Understanding chronic ocular surface pain: An unmet need for targeted drug therapy. Ocul Surf. 2022; 26:148-56.
16. Patel S, Hwang J, Mehra D, Galor A. Corneal nerve abnormalities in ocular and systemic diseases. Exp Eye Res. 2021; 202:108284.
17. Grueb M, Wallenfels-Thilo B, Denk O, Mielke J, Reinthal E, Rohrbach J M, et al. Monoamine receptors in human corneal epithelium and endothelium. Acta Ophthalmol Scand. 2006; 84(1):110-5.
19. Potrebic S, Ahn A H, Skinner K, Fields H L, Basbaum A I. Peptidergic nociceptors of both trigeminal and dorsal root ganglia express serotonin 1D receptors: implications for the selective antimigraine action of triptans. J Neurosci. 2003; 23(34):10988-97.
20. Veiga Moreira T H, Gover T D, Weinreich D. Electrophysiological properties and chemosensitivity of acutely dissociated trigeminal somata innervating the cornea. Neurosci. 2007; 148(3):766-74.
21. Millan M J, Maiofiss L, Cussac D, Audinot V, Boutin J A, Newman-Tancredi A. Differential actions of antiparkinson agents at multiple classes of monoaminergic receptor. I. A multivariate analysis of the binding profiles of 14 drugs at 21 native and cloned human receptor subtypes. J Pharmacol Exp Ther. 2002; 303(2):791-804.
22. Newman-Tancredi A, Cussac D, Audinot V, Nicolas J P, De Ceuninck F, Boutin J A, et al. Differential actions of antiparkinson agents at multiple classes of monoaminergic receptor. II. Agonist and antagonist properties at subtypes of dopamine D2-like receptor and α₁/α₂-adrenoceptor. J Pharmacol Exp Ther. 2002; 303(2):805-14.
23. Newman-Tancredi A, Cussac D, Quentric Y, Touzard M, Verrièle L, Carpentier N, et al. Differential actions of antiparkinson agents at multiple classes of monoaminergic receptor. III. Agonist and antagonist properties at serotonin, 5-HT₁ and 5-HT₂, receptor subtypes. J Pharmacol Exp Ther. 2002; 303(2):815-22.
24. Ohta K, Kuno S, Mizuta I, Fujinami A, Matsui H, Ohta M. Effects of dopamine agonists bromocriptine, pergolide, cabergoline, and SKF-38393 on GDNF, NGF, and BDNF synthesis in cultured mouse astrocytes. Life Sci. 2003; 73(5):617-626. https://doi.org/10.1016/s0024-3205(03)00321-7.
25. Martin X D, Brennan M C. Serotonin in human tears. Eur J Ophthalmol. 1994; 4(3):159-65.
26. Tashiro A, Okamoto K, Chang Z, Bereiter D A. Behavioral and neurophysiological correlates of nociception in an animal model of photokeratitis. Neurosci. 2010; 169(1):455-62.
28. Bendele A M, Spaethe S M, Benslay D N, Bryant H U. Anti-inflammatory activity of pergolide, a dopamine receptor agonist. J Pharmacol Exp Ther. 1991; 259(1):169-175.
29. Uberti D, Piccioni L, Colzi A, Bravi D, Canonico P L, Memo M. Pergolide protects SH-SY5Y cells against neurodegeneration induced by H₂O₂. Eur J Pharmacol. 2002; 434(1-2):17-20.
30. Wing L, Lin M C. Sex disparity in how pain sensitivity influences dry eye symptoms. Cornea. 2019; 38(10): 1291-1298.
31. Roper-Hall M J. Thermal and chemical burns. Trans Ophthalmol Soc U K. 1965; 85: 631-53.
32. Hayreh S S, Piegors D J, Heistad D D. Serotonin-induced constriction of ocular arteries in atherosclerotic monkeys: Implications for ischemic disorders of the retina and optic nerve head. Arch Ophthalmol. 1997; 115(2):220-228.
33. Seiple W, Jennings D, Rosen R B, Borchert L, Canale L, Fagan N, et al. Ophthalmologic baseline characteristics and 2-year ophthalmologic safety profile of pramipexole I R compared with ropinirole I R in patients with early Parkinson's disease. Parkinson's Dis. 2016; 2016:1-14.
34. Dua H S, Ting D S, Al Saadi A, Said D G. Chemical eye injury: Pathophysiology, assessment and management. Eye. 2020; 34(11):2001-19.
35. Aloe L, Tirassa P, Lambiase A. The topical application of nerve growth factor as a pharmacological tool for human corneal and skin ulcers. Pharmacol Res. 2008; 57(4):253-8.
36. Ergen S K, Subasi S, Rencber S F, Duruksu G, Yazir Y. Evaluation of clinical and histological effects of KGF-2 and NGF on corneal wound healing in an experimental alkali burn rabbit model. Exp Eye Res. 2022; 223:109190.
37. Xiang Y, Zhou W, Wang P, Yang H, Gao F, Xiang H, et al. Alkali burn induced corneal spontaneous pain and activated neuropathic pain matrix in the central nervous system in mice. Cornea. 2017; 36(11):1408-14.
40. Habibi R N, Lee M D. Treatment of dry eye from laser-assisted in situ keratomileusis with recombinant human nerve growth factor (cenegermin). Cornea. 2021; 40(8):1059-61.
42. Siegl C, König-Schuster M, Nakowitsch S, Koller C, Graf P, Unger-Manhart N, et al. Pharmacokinetics of topically applied tacrolimus dissolved in Marinosolv, a novel aqueous eye drop formulation. Eur J Pharm Biopharm. 2019; 134:88-95.
43. Mogil J S. Qualitative sex differences in pain processing: Emerging evidence of a biased literature. Nat Rev Neurosci. 2020; 21(7):353-65.
44. Vasilopoulos T, Wardhan R, Rashidi P, Fillingim R B, Wallace M R, Crispen P L, et al. Patient and procedural determinants of postoperative pain trajectories. Anesthesiology. 2021; 134(3):421-34.
45. Sacchetti M, Lambiase A, Schmidl D, Schmetterer L, Ferrari M, Mantelli F, Allegretti M, Garhoefer G. Effect of recombinant human nerve growth factor eye drops in patients with dry eye: A phase IIa, open label, multiple-dose study. Br J Ophthalmol. 2020; 104(1):127-35.
46. Coassin M, Lambiase A, Costa N, De Gregorio A, Sgrulletta R, Sacchetti M, Aloe L, Bonini S. Efficacy of topical nerve growth factor treatment in dogs affected by dry eye. Graefes Arch Clin Exp. 2005; 243(2):151-5.
47. Gong Q, Zhang S, Jiang L, Lin M, Xu Z, Yu Y, et al. The effect of nerve growth factor on corneal nerve regeneration and dry eye after LASIK. Exp Eye Res. 2021; 203:108428.
48. Joo M J, Yuhan K R, Hyon J Y, Lai H, Hose S, Sinha D, et al. The effect of nerve growth factor on corneal sensitivity after laser in situ keratomileusis. Arch Ophthalmol. 2004; 122(9):1338-41.
50. Francis P C, Carlson K H, Owen N V, Adams E R. Preclinical toxicology studies with the new dopamine agonist pergolide. Acute, subchronic, and chronic evaluations. Drug Res. 1994; 44(3):278-84.
51. Gilbert D L, Sethuraman G, Sine L, Peters S, Sallee F R. Tourette's syndrome improvement with pergolide in a randomized, double-blind, crossover trial. Neurology. 2000; 54(6):1310-5.
52. Simonds S E, Cowley M A. Speed-dieting: dopamine agonists promote weight loss. Nat Metab. 2019; 1(9):851-2.
53. Beykin G, Stell L, Halim M S, Nuilez M, Popova L, Nguyen B T, et al. Phase 1b randomized controlled study of short course topical recombinant human nerve growth factor (rhNGF) for neuroenhancement in glaucoma: Safety, tolerability, and efficacy measure outcomes. Am J Ophthalmol. 2022; 234:223-34.
54. Nguyen D H, Beuerman R W, Thompson H W, DiLoreto D A. Growth factor and neurotrophic factor mRNA in human lacrimal gland. Cornea. 1997; 16(2):192-9.
55. Droogmans S, Roosens B, Cosyns B, Degaillier C, Hernot S, Weytjens C, Garbar C, Caveliers V, Pipeleers-Marichal M, Franken P R, Lahoutte T. Cyproheptadine prevents pergolide-induced valvulopathy in rats: An echocardiographic and histopathological study. Am J Physiol Heart Circ. 2009; 296(6):H1940-8.
56. Cardiff R D, Miller C H, Munn R J. Manual hematoxylin and eosin staining of mouse tissue sections. Cold Spring Harb Protoc. 2014; 2014(6):pdb-rot073411.
57. McCann P, Abraham A G, Mukhopadhyay A, Panagiotopoulou K, Chen H, Rittiphairoj T, et al. Prevalence and incidence of dry eye and meibomian gland dysfunction in the United States: A systematic review and meta-analysis. JAMA Ophthalmol. 2022; 140(12):1181-1192.
58. Daeschler S C, Mirmoeini K, Gordon T, Chan K, Zhang J, Ali A, et al. Sustained release of tacrolimus from a topical drug delivery system promotes corneal reinnervation. Transl Vis Sci Technol. 2022; 11(8):20.
59. Akhtar R A. Effects of norepinephrine and 5-hydroxytryptamine on phosphoinositide-PO₄ turnover in rabbit cornea. Exp Eye Res. 1987; 44(6):849-62.
60. Crider J Y, Williams G W, Drace C D, Katoli P, Senchyna M, Sharif N A. Pharmacological characterization of a serotonin receptor (5-HT7) stimulating cAMP production in human corneal epithelial cells. Investig Ophthalmol Vis Sci. 2003; 44(11):4837-44.
61. Grueb M, Rohrbach J M, Schlote T, Mielke J. Serotonin (5-HT7) receptor-stimulated activation of cAMP-PKA pathway in bovine corneal epithelial and endothelial cells. Ophthal Res. 2012; 48(1):22-7.
62. Klyce S D, Palkama K A, Härkönen M, Marshall W S, Huhtaniitty S, Mann K P, et al. Neural serotonin stimulates chloride transport in the rabbit corneal epithelium. Investig Ophthalmol Vis Sci. 1982; 23(2):181-92.
63. Alvarez L J, Turner H C, Zamudio A C, Candia O A. Serotonin-elicited inhibition of Cl⁻ secretion in the rabbit conjunctival epithelium. Am J Physiol Cell Physiol. 2001; 280(3):C581-92.

It will be apparent that the precise details of the methods or compositions described may be varied or modified without departing from the spirit of the described aspects of the disclosure. We claim all such modifications and variations that fall within the scope and spirit of the claims below.

We claim:

1. A method of treating an ocular chemical burn, comprising administering an effective amount of pergolide to an eye of a subject having the ocular chemical burn.

2. The method of claim 1, further comprising selecting the subject having the ocular chemical burn.

3. The method of claim 1, wherein the pergolide is administered as a pergolide solution via eye drop.

4. The method of claim 3, wherein the effective amount of the pergolide solution administered is a drop of about 25 μl to about 60 μl;
about 5 μl to about 7 μl on the cornea; or
about 7 μl to about 10 μl on the eyelid fornix.

5. The method of claim 4, wherein the concentration of pergolide in the pergolide solution is about 50 μg/ml to about 600 μg/ml.

6. The method of claim 5, wherein the concentration of pergolide in the pergolide solution is about 300 μg/ml.

7. The method of claim 5, wherein the concentration of pergolide in the pergolide solution is about 600 μg/ml.

8. The method of claim 3, wherein administering the effective amount of the pergolide solution does not result in cardiac valvulopathy in the subject.

9. The method of claim 8, wherein administering the effective amount of the pergolide solution does not result in valvular fibrosis, significant cardiac glycosaminoglycan deposits, or cardiac myxoid changes in the subject.

10. The method of claim 3, wherein the eye drop is administered 3 times a day, 2 times a day, or 1 time a day.

11. The method of claim 1, wherein the ocular chemical burn is caused by an alkali compound.

12. The method of claim 1, wherein administering the effective amount of pergolide reduces corneal haze.

13. The method of claim 1, wherein administering the effective amount of pergolide increases central corneal nerve fiber density.

14. The method of claim 13, wherein the central corneal nerve fiber density is increased within 3 weeks following the administering.

15. The method of claim 1, wherein administering the effective amount of pergolide prevents a decrease in limbal nerve density.

16. The method of claim 15, wherein the limbal nerve density is increased within 3 weeks following the administering compared to a control.

17. The method of claim 1, wherein the subject did not previously receive ocular nerve grafting.

18. The method of claim 1, further comprising administering an effective amount of a cycloplegic, a NSAID, a steroid, vitamin C, an antibiotic, lubricating eye drops, doxycycline, a tetracycline derivative, a non-aminoglycoside ocular antibiotic, erythromycin, a fluoroquinolone, mitomycin C, a medroxprogesterone eyedrop, a amniotic membrane eyedrop, an autologous/allogenic eye drop, a citrate eye drop, or an ascorbate eye drop to the eye of the subject having the ocular chemical burn.

19. The method of claim 3, wherein the pergolide solution comprises hyaluronate or hydroxypropyl methylcellulose.

20. A kit, comprising a pergolide solution contained within a container configured to dispense a measured dosage of the pergolide solution and instructions for use.
